(19) United States
(12) Patent Application Publication
(10) Pub. No.: US 2021/0366619 A1
(43) Pub. Date: Nov. 25, 2021

(54) RECOVERY PROFILE CLUSTERING TO DETERMINE TREATMENT PROTOCOL AND PREDICT RESOURCING NEEDS

(71) Applicant: KONINKLIJKE PHILIPS N.V., Eindhoven (NL)

(72) Inventors: Xia CHEN, Lorton, VA (US); Gaurav TRIVEDI, Pittsburgh, PA (US); Evan Joel KASEMAN, Monroeville, PA (US); Dirk Ernest VON HOLLEN, Clark, NJ (US); Sara KRON, Pittsburgh, PA (US)

(21) Appl. No.: 17/218,316

(22) Filed: Mar. 31, 2021

Related U.S. Application Data
(60) Provisional application No. 63/027,406, filed on May 20, 2020.

(51) Int. Cl. G16H 50/30; G16H 50/80; G16H 10/60; G16H 20/10; A61B 5/00

(52) U.S. Cl. CPC G16H 50/30; G16H 50/80; A61B 5/4836; G16H 20/10; A61B 5/7275; G16H 10/60

(57) ABSTRACT

An apparatus and method involve not just comparing a patient's recovery but improving predications by grouping the patient into a phenotype to trend the patient's rate of progress compared to existing phenotype clusters to predict, based on a comparison of rate of progress of decline, and assess in order to benefit in the improvement of allocation and timing of future resource needs, including the economic impact.

### CROSS-REFERENCE TO PRIOR APPLICATIONS

This application claims the benefit of U.S. Provisional Application No. 63/027,406, filed on 20 May 2020. This application is hereby incorporated by reference herein.

### BACKGROUND OF THE INVENTION

### 1. Field of the Invention

The present invention pertains to assisting healthcare institutions in determining the ongoing status of a patient's recovery journey during a pandemic and, in particular, to an apparatus and method for improving predications by grouping the patient into various phenotypes to trend each patient's rate of progress compared with existing phenotype clusters to improve the allocation and timing of future resource needs and economic impact.

### 2. Description of the Related Art

Recent history has seen a handful of infectious respiratory borne illnesses. This includes the 2003 Severe Acute Respiratory Syndrome (SARS), 2014 Middle-Eastern Respiratory Syndrome (MERS), and the more recent severe acute respiratory syndrome coronavirus 2 (SARS-CoV-2). The COVID-19 disease has spread to over 100 countries worldwide and has reached pandemic proportions. Remuzzi, A. & Remuzzi, G. COVID-19 and Italy: what next? The Lancet (2020) doi:10.1016/S0140-6736(20)30627-9. This virus outbreak originated in Wuhan, China in December 2019 and rapidly spread to other regions of the world in a matter of a few weeks and, as of Apr. 29, 2020, it has infected many millions of people worldwide causing many hundreds of thousands of deaths. Coronavirus Update (Live): 1,411,348 Cases and 81,049 Deaths from COVID-19 Virus Pandemic—Worldometer https://www.worldometers.info/coronavirus/; Dong, E., Du, H. & Gardner, L. An interactive web-based dashboard to track COVID-19 in real time. Lancet Infect. Dis. 0, (2020).

The symptoms of these acute respiratory illnesses, such as COVID-19, may vary from being very mild, and can include a fever, cough, headache and sore throat, chills and shortness of breath, to severe respiratory symptoms. COVID-19: How can I protect myself? Mayo Clinic https://www.mayoclinic.org/diseases-conditions/coronavirus/expert-answers/novel-coronavirus/faq-20478727; Xu, Z. et al. Pathological findings of COVID-19 associated with acute respiratory distress syndrome. Lancet Respir. Med. 8, 420-422 (2020). Some patients may also feel asymptomatic and act as unsuspecting carriers of the disease. Chan, J. F.-W. et al. A familial cluster of pneumonia associated with the 2019 novel coronavirus indicating person-to-person transmission: a study of a family cluster. The Lancet 395, 514-523 (2020). More critical patients may experience acute respiratory inflammations requiring clinical attention. These patients are often put on mechanical ventilation as their lungs recover from the virus and are unable to perform regular breathing functions.

The viruses causing these respiratory diseases can spread very rapidly. Thus, such pandemics may quickly overwhelm the healthcare systems, which may find it difficult to keep up with large volumes of patients requiring testing, monitoring, and hospitalization. This also presents several challenges in overall management of the disease in large populations. Apart from tracking the spread of the disease, it is important to make epidemiological predictions as well as public health decisions for countries affected using dashboards and analytics methods. An interactive web-based dashboard to track COVID-19 in real time, Id. Self-reporting and smart wearable sensing of symptoms in the population can both help patients monitor their condition and give insights to the healthcare system, such as the use of wearable data for tracking COVID-19 symptoms. Reuter, E. Scripps researchers tap wearable data to track Covid-19 and flu, MedCity News https://medcitynews.com/2020/03/scripps-researchers-tap-wearable-data-to-track-covid-19-and-flu/(2020); Predicting coronavirus? SF emergency workers wear state-of-the-art rings in new study. SFChronicle.com https://www.sfchronicle.com/bayarea/article/Predicting-coronavirus-SF-emergency-workers-wear-15149729.php (2020). Advanced remote monitoring systems can also help in monitoring those under recovery, such as after being extubated from a ventilator and being discharged from the hospital.

Previous solutions have revolved around patient deterioration detection (as described in WO 2012/117316) and have focused on the ability to assess the patient change back to baseline. Dynamic risk manage systems have focused on calculating a risk level by continuously receiving physiological real time data to revise a score level and by comparing risk level (as described in WO 2013/116243). Nevertheless, improvements in treatment in the event of a pandemic and otherwise would be desirable.

### SUMMARY OF THE INVENTION

Accordingly, it is an object of the present invention to provide an improved apparatus and method for improving healthcare institutions in determining the ongoing status of a patient's recovery journey and the impact to future resources, both material and financial, that overcomes the shortcomings of conventional systems and methods for providing and assessing treatment. This object is achieved according to one embodiment of the present invention by providing an apparatus and method that involve not just comparing a patient's recovery but improving predications by grouping each patient into a phenotype to trend the patient's rate of progress compared to existing phenotype clusters to predict, based on a comparison of rate of progress or decline, and assess in order to benefit in the improvement of allocation and timing of future resource needs, including the economic impact.

While a large number of people may be infected by the virus, the majority have mild symptoms that can be managed at home. Those who undergo hospitalization also require sustained monitoring after being discharged. A significant need is to identify patients that require close monitoring and management, both at home and in a hospital setting. Hospital systems must be prepared to rapidly address the resourcing requirements (both staff and equipment) to address the massive influx of patients in a pandemic-like scenario. Advanced patient monitoring support greatly improves the ability of the hospital systems to free up resources and better manage pandemic levels of incoming patients.

The system and method of the disclosed and claimed concept advantageously enhance remote patient monitoring ability by clustering the patient based upon a phenotype that is established upon discharge as a baseline to compare trends and recovery rates received at regular intervals based on clusters that each include large numbers of patients. The system and method of the disclosed and claimed concept also advantageously enable large number of patients with similar care plans to be monitoring and tracked and to have their progress assessed to determine if it is adequate or if changes in medical resources or care are indicated based upon outliers.

The disclosed and claimed concept advantageously leverages physiological signals, subjective inputs, and information from Electronic Medical Record (EMR) and/or Electronic Health Record (EHR) data to create a patient recovery profile at discharge, to identify an appropriate recovery phenotype and identify outliers, and to derive a triage risk score and a rate of recovery. This information guides the choice of treatment protocol and predicts the resource needs that will be required of the healthcare providers. Ultimately, patients will receive better care during their recovery journey.

Accordingly, aspects of the disclosed and claimed concept are provided by an improved method of prescribing a treatment protocol for each of a plurality of patients, the general nature of which can be stated as: at each of a plurality of times: for each patient of the plurality of patients: detecting a number of risk factors of the patient, determining a triage risk index of the patient based at least in part upon an at least partial summation of at least a subset of the number of risk factors of the patient, and determining a recovery rate of the patient based at least in part upon a rate of change in the triage risk index of the patient, grouping together each patient from among the plurality of patients into a subset from among a plurality of subsets, each subset of the plurality of subsets corresponding with a phenotype from among a plurality of phenotypes, each phenotype from among the plurality of phenotypes corresponding with a treatment protocol from among a plurality of treatment protocols, the grouping being based at least in part upon a recovery profile of each patient in the subset, and prescribing for each patient the treatment protocol that corresponds with the phenotype that corresponds with the subset to which the patient is assigned.

Other aspects of the disclosed and claimed concept are provided by an improved apparatus structured to prescribe a treatment protocol for each of a plurality of patients and that includes a processor and a storage, the storage having stored therein a number of routines which, when executed on the processor, cause the apparatus to perform a number of operations, the general nature of which can be stated as: at each of a plurality of times: for each patient of the plurality of patients: detecting a number of risk factors of the patient, determining a triage risk index of the patient based at least in part upon an at least partial summation of at least a subset of the number of risk factors of the patient, and determining a recovery rate of the patient based at least in part upon a rate of change in the triage risk index of the patient, grouping together each patient from among the plurality of patients into a subset from among a plurality of subsets, each subset of the plurality of subsets corresponding with a phenotype from among a plurality of phenotypes, each phenotype from among the plurality of phenotypes corresponding with a treatment protocol from among a plurality of treatment protocols, the grouping being based at least in part upon a recovery profile of each patient in the subset, and prescribing for each patient the treatment protocol that corresponds with the phenotype that corresponds with the subset to which the patient is assigned. As employed herein, the expression “a number of” and variations thereof shall refer broadly to any non-zero quantity, including a quantity of one.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic depiction of an improved apparatus in accordance with an aspect of the disclosed and claimed concept;

FIG. 2 depicts an improved method in accordance with the disclosed and claimed concept;

FIG. 3 is a depiction of a set of Cartesian coordinates upon which is plotted a location, based upon the selected parameters of the recovery profile, for each patient from among a number of patients that together constitute a subset from among a plurality of patients;

FIG. 4 is another depiction of the set of Cartesian coordinates upon which is plotted a location, based upon the selected parameters of the recovery profile, for each patient from among another number of patients that constitute another subset from among the plurality of patients.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

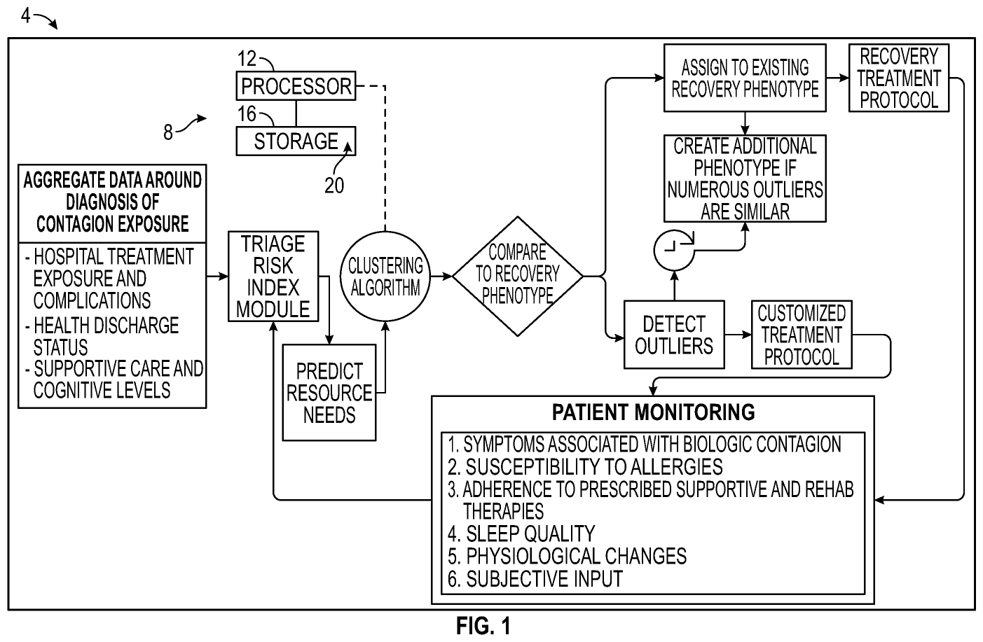

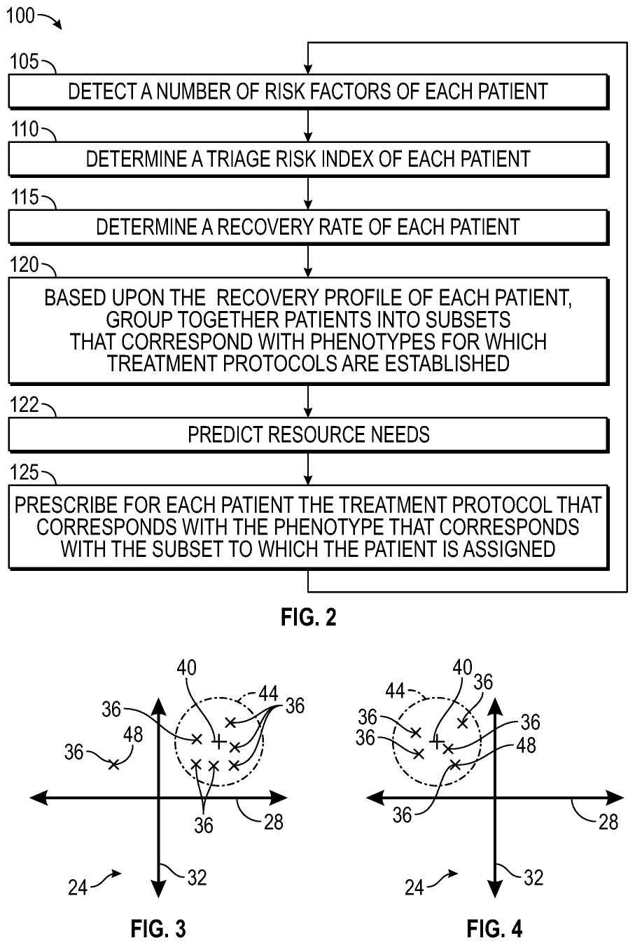

FIG. 1 illustrates the overall architecture of the solution proposed in the disclosed and claimed concept. More specifically, an improved apparatus 4 in accordance with the disclosed and claimed concept is depicted in a schematic fashion in FIG. 1. Apparatus 4 can be employed in performing an improved method 100 that is likewise in accordance with the disclosed and claimed concept and at least a portion of which is depicted in a schematic fashion in FIG. 2. Apparatus 4 can be characterized as including a processor apparatus 8 that can be said to include a processor 12 and a storage 16 that are connected with one another. Storage 16 is in the form of a non-transitory storage medium that has stored therein a number of routines 20 that are likewise in the form of a non-transitory storage medium and that include instructions which, when executed on processor 12, cause apparatus 4 to perform certain operations such as are mentioned elsewhere herein.

A triage risk index to baseline condition for each patient is calculated at discharge based on background information of comorbidities, hospital treatment exposure and complications, and health discharge status along with supportive care and cognitive factors, along with the patient monitoring data (see FIG. 1). Utilizing this aggregate data, an algorithm embodied in the number of routines 20 assigns the person to an appropriate recovery phenotype from among a plurality of recovery phenotypes by comparing the patient's parameters, i.e., a recovery profile for the patient that is based at least in part upon the patient's triage risk index and a recovery rate, with the various existing phenotypes. If a match is not made, the system 4 customizes the treatment protocol, or indicates that individualized medical care is to be given to the patient, or takes other action to best address the symptoms of that patient.

Parameters that the apparatus 4 monitors during recovery include, but are not limited to, factors and status related not only physiological symptoms such as biologic contagion status (mild to severe), susceptibility to allergies based on environmental factors, preexisting medical conditions, complications from extended hospitalization, adherence to prescribed supportive and rehabilitative therapies, sleep quality, level of activity changes to routine challenges, engagement and factors that cannot be measured objectively from interaction with questionnaires or surveys or requests for testing, and differential from normal thresholds of similar recovery cluster dynamically based on parameters. From this data, the efficacy of the recovery treatment protocol can be evaluated, and modifications can be made based on the appropriate recovery phenotype that aligns with current parameters if the routines 20 dynamically detect a differential from normal thresholds of that recovery profile. If the routines 20 designate enough people as outliers from the existing recovery phenotypes, it will create a new phenotype to cluster these patients and implement the most effective recovery treatment protocol.

As demonstrated in the COVID-19 pandemic, the situation is dynamic, and conditions change rapidly. To best account for the constant change, the routines 20 employ machine learning and build machine learning capabilities as more data is gathered from the population of patients, thereby establishing more distinct thresholds for the recovery phenotypes and enhancing the performance of the apparatus 4.

Patient Monitoring Through Integrated Information

The disclosed and claimed concept builds an intelligent patient monitoring apparatus 4 with the capability of extracting patient recovery insights through continuous monitoring and analyzing various information from the patient. These insights will inform either the selection of the appropriate patient-provider treatment protocol or creation of a new treatment protocol. The apparatus 4 thus advantageously provides adaptive recommendations of the optimum treatment protocol to the providers during a patient recovery journey. That is, each recovery phenotype has a corresponding treatment protocol, and the assignment of a patient to a particular recovery phenotype results in the treatment protocol that corresponds with that particular recovery phenotype being prescribed for that patient.

Ultimately, apparatus 4 advantageously alleviates pressure on healthcare providers by enhancing and establishing a plurality of standard treatment protocols, which advantageously enables the healthcare providers to easily integrate these treatment protocols into their workflow. Meanwhile, apparatus 4 improves the experience of the patients on their recovery journey by identifying and providing enhanced treatment protocols and customized treatment protocols, when appropriate.

Various categories of information sources can be used in apparatus 4 such as real-time physiological signals from sensors (e.g. sleep quality, vitals), subjective inputs from patients (e.g. digital questionnaires, simple query/response), and information from EMR systems (e.g. health history, hospital treatments and complications, prescribed therapies, adherence and outcomes).

Estimation of Triage Risk and Recovery Rate

The disclosed and claimed concept defines a triage risk index as a function of various parameters from a patient recovery profile as shown in FIG. 1. Specifically, this index can be estimated based on the following information.

Assume R(t)={r₀(t), r₁(t) . . . , r_{N}(t)} as a patient recovery profile at time t, where r₀(t), r₁(t) . . . , r_{N}(t) each represent a quantified positive integer risk measurement of each parameter, as illustrated in FIG. 1. The value range of each parameter is determined by the prior knowledge of percentage of impact on the total triage risk index. For example, if the adherence parameter has more impact than the susceptibility to allergies, its value range will be larger. The low end of the range corresponds to the lowest risk.

Assume R(0) as the baseline recovery profile at discharge.

Assume T_{RI}(t) is the triage risk index at time t and it is defined as the summation of the elements in R(t): T_{RI}(t)=Σᵢ₌₁^{N}(rᵢ(t))

The triage risk index can be used as one of the contributing factors in determining the need of a customized treatment protocol.

Assume RR(t) is the recovery rate at time t and it is defined as the derivative of the triage risk index: RR(t)=dT_{RI}(t)/dt

It informs the resource needs and is representative of the rate of change of the triage risk index at time t.

R(t), T_{RI}(t) and RR(t) will be calculated at discharge of a patient and will also be estimated in a predefined or selected frequency (e.g. daily) during the recovery journey of the patient. For instance, data for a given patient can be collected daily, hourly, continuously, etc., and the aforementioned calculations of R(t), T_{RI}(t) and RR(t) can be performed with similar or different frequency.

Recovery Phenotype Creation and Membership Assessment Criteria

The disclosed and claimed apparatus 4 and method 100 advantageously create a set of recovery phenotypes through initially clustering the recovery profiles of the various patients according to the corresponding treatment protocols. In other words, all the recovery profiles sharing the same treatment protocol will be classified into one cluster. The exemplary recovery profile that is employed herein is described as being based at least in part upon the triage risk index and the recovery rate. It is nevertheless understood that the recovery profile can be, and likely will be, based upon numerous additional factors, such as the risk measures of adherence parameter, the susceptibility to allergies, etc. However, for the sake of simplicity of disclosure the recovery profile that is described in connection with FIGS. 3 and 4 is described in terms of the risk measures of adherence parameter and the susceptibility to allergies, both of which are indicated along a corresponding axis of a set of Cartesian coordinates. This also could be done instead with any two parameters from the recovery profile on the two axes or, by way of further example, the risk measures of the symptoms and the sleep quality could be added to the risk measures of adherence parameter and the susceptibility to allergies such that the recovery profile is based upon four factors.

For example, and as is shown in FIG. 3, a set of Cartesian coordinates 24 can be defined, with an abscissa 28 that is representative of the risk measures of adherence and an ordinate 32 that is representative of the susceptibility to allergies. It is reiterated, however that real world recovery profiles are going to have many dimensions for their risk index, and the instant document is illustrating this on a 2D plot for the sake of simplicity. One can extrapolate this example to a hypothetical multi-dimension plot.

For any given patient, the patient's the risk measures of the symptoms and the sleep quality can be plotted along the abscissa 28 and ordinate 32, respectively, to result in a location 36 on the set of Cartesian coordinates 24 that is representative of the current recovery status of the patient. FIG. 3 shows a plurality of such locations 36 that each correspond with a patient that has been assigned to a given recovery phenotype.

For each cluster, a recovery phenotype can be determined by finding the representative recovery profile of this cluster. This can be done, for example, by calculating a centroid 40 of all the recovery profiles in this cluster, as is shown in FIG. 3. As both updated recovery profiles and new recovery profiles are input to the apparatus 4, each phenotype will be updated and will reflect the typical recovery profile more accurately for each cluster.

If there are total L treatment protocols, there will be total L recovery phenotypes created accordingly. To assess if a new recovery profile belongs to one of the existing phenotypes, it is necessary to create a metric and set up an acceptance criterion for membership.

For each phenotype, a maximum radius 44 on the set of Cartesian coordinates 24 between any locations 36 (i.e., locations 36 on the set of Cartesian coordinates 24 that are representative of patient recovery statuses) and the centroid 40 of the cluster, i.e. recovery phenotype, can be used as such a metric. In some embodiments, a dispersion index can be used as a metric. The maximum radius 44 can be determined in any of a number of fashions and potentially could be determined by assessing the extent to which the assignment of patients to a particular recovery phenotype and the resultant prescribing of the corresponding treatment to those patients results in an overall desirable therapeutic outcome. For instance, if it is determined that an excessive number of patients were assigned to a given recovery phenotype are not responding to the prescribed treatment and eventually are assigned to other recovery phenotypes, this might suggest that the maximum radius 44 is too great and should be reduced. This can be optimized via machine learning which is mentioned elsewhere herein and which is employed in conjunction with the improved apparatus 4 and the improved method 100.

Based on the established phenotypes and their character, the following Look-Up-Table (LUT) (Table 1) can be built for using in the process of assessing a new recovery profile:

TABLE 1
Look Up Table

| Phenotype | Maximum Radius | Treatment protocol |
|---|---|---|
| C₁ | δ₁ | P₁ |
| C₂ | δ₂ | P₂ |
| . . . | . . . | . . . |
| C_{L} | δ_{L} | P_{L} |

Where C₁, C₂ . . . , C_{L} are the recovery phenotypes created and P₁, P₂ . . . , P_{L} are the corresponding treatment protocols.

Membership Qualification Evaluation of a New Recovery Profile

The disclosed and claimed concept advantageously determines if a new recovery profile would fit into any of the existing recovery phenotypes through the following approach, which can be said to include k-means/clustering:

1) Assume R is the new recovery profile;

2) Assume Ci is the ith phenotype;

3) Calculate the Euclidean distance between R and Ci;

4) Check to see if the distance calculated is less than δi by using the LUT. If yes, save the Ci along with the distance calculated in the set of candidate recovery phenotypes. If no, a new phenotype creation might be considered if the triage risk index of R is higher than (e.g. a threshold can be set) those from all the existing phenotypes.

5) Repeat steps 2 to 4 for i=1, 2 . . . , L;

6) From the candidate recovery phenotype, choose the one with the shortest distance from R as its phenotype.

Adaptive Treatment Protocol Recommendation

In the preferred embodiment, the disclosed and claimed concept advantageously determines and recommends to a provider the appropriate treatment protocol for each patient at discharge and during the recovery journey. Based on the approach discussed elsewhere herein, if the recovery profile R of a patient fits into one of the existing phenotype, the corresponding treatment protocol can be found from the LUT above.

To further assess if a new phenotype needs to be created, the need for a new treatment protocol is evaluated. In fact, a new treatment protocol will be required and created only if the major contribution parameter(s) from the recovery profile can be improved, such as, supportive care adherence. In this case, R is a detected outlier and it becomes of center of a new cluster (i.e. new phenotype) along with the new treatment protocol. If a new treatment protocol is not warranted, R will be added into the nearest cluster and use its treatment protocol. In some embodiment, R might not be added into the nearest cluster, but use its treatment protocol. In some embodiments, the apparatus 4 provides the recommendations to the providers via dashboard on patient portals or providers' mobile devices.

By way of further example, FIG. 3 depicts at the numeral 48 one location 36 (36 represents the existing patient profiles in a cluster) of a patient that lies outside the maximum radius 44 of the recovery phenotype that is depicted in the upper right quadrant of the set of Cartesian coordinates 24 of FIG. 3. This patient would therefore be considered to be an outlier. However, FIG. 4 depicts another recovery phenotype different than the recovery phenotype of FIG. 3 and in which the same location 36, 48 lies within the maximum radius 44 of the other phenotype that is depicted in FIG. 4. That is, whereas the patient represented by location 36, 48 was considered to be in outlier with regard to recovery phenotype of FIG. 3 because its Euclidean position was outside the maximum radius of the phenotype of FIG. 3, the same patient was able to be assigned to a different recovery phenotype that is depicted in FIG. 4 where the location 36, 48 of the same patient lies within the maximum radius 44 of that other phenotype. This patient would therefore be prescribed the treatment protocol that corresponds with the phenotype that is depicted in FIG. 4.

Prediction of Resource Needs

In the preferred embodiment, this invention forecasts the resource needs (e.g. supportive therapy devices, personnel) based on the recovery rates of all the phenotypes. The recovery rate of each phenotype can be estimated from the recovery rates of patients in the same cluster. The average recovery rate of the patients in the same cluster can be used. By way of example, the apparatus 4 provides the resource forecasts to the providers via dashboard on patient portals. For instance, apparatus 4 an improved method 4 can be used to predict resource needs, staffing needs, and other types of needs.

The improved method 100 in accordance with the disclosed and claimed concept is depicted generally in FIG. 2. Processing begins, as at 105, with the detecting at a given time of a number of risk factors of each patient. Processing continues, as at 110, with the determining of a triage risk index of each patient at the given time. The triage risk index can be a simple summation of the risk factors or can include some weighting or other type of partial summation of the risk factors. The recovery rate of each patient is then determined, as at 115, it being understood that the recovery rate is the derivative or the rate of change of the triage risk index of the patient at the given time.

Processing continues, as at 120 where, based upon the recovery profile of each patient, the patients are grouped together into subsets that correspond with phenotypes for which treatment protocols are established. As noted, the recovery profile can be based at least in part upon the triage risk index, the recovery rate, the risk measures of adherence, the susceptibility to allergies, etc., in any combination, and without limitation. Processing continues, as at 122, with the predicting of resource needs which can be, for example, the predicting of staffing needs, resource needs, equipment needs, etc. Processing then continues, as of 125, with the prescribing for each patient of the treatment protocol that corresponds with the phenotype that corresponds with the subset to which the patient is assigned.

It is noted that processing thereafter continues at 105 where the process is repeated periodically in order to optimize the assignment of the various patients to the various phenotypes in order to optimize the overall treatment of patients. By optimizing the treatment of the patients, individual treatment resources that would otherwise be overwhelmed in a pandemic situation can be used to their greatest value by grouping patients having a similar triage risk index and a similar recovery rate into the phenotypes for treatment according to the same treatment protocol. This avoids the need for individualized medical treatment for each patient and thus advantageously enables hospitals and other treatment facilities with limited resources to effectively treat the large number of patients that exist in a pandemic situation. Other benefits will be apparent.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method of prescribing a treatment protocol for each of a plurality of patients, comprising:
at each of a plurality of times:
for each patient of the plurality of patients:
detecting a number of risk factors of the patient;
determining a triage risk index of the patient based at least in part upon an at least partial summation of at least a subset of the number of risk factors of the patient; and
determining a recovery rate of the patient based at least in part upon a rate of change in the triage risk index of the patient;
grouping together each patient from among the plurality of patients into a subset from among a plurality of subsets, each subset of the plurality of subsets corresponding with a phenotype from among a plurality of phenotypes, each phenotype from among the plurality of phenotypes corresponding with a treatment protocol from among a plurality of treatment protocols, the grouping being based at least in part upon a recovery profile of each patient in the subset; and
prescribing for each patient the treatment protocol that corresponds with the phenotype that corresponds with the subset to which the patient is assigned.

2. The method of claim 1, wherein the grouping further comprises:
making a determination, for a particular patient of the plurality of patients who is in a particular subset of the plurality of subsets, and based at least in part upon the recovery profile of the particular patient, that the particular patient does not belong in the particular subset;
assigning the particular patient to another subset of the plurality of subsets, the another subset being different from the particular subset, based at least in part upon the recovery profile of the particular patient; and
prescribing for the particular patient the treatment protocol that corresponds with the phenotype that corresponds with the another subset.

3. The method of claim 2, wherein the grouping further comprises:
assigning to each patient in the particular subset a location along a set of Cartesian coordinates that corresponds with the recovery profile of the patient;
determining a centroid of the locations of the patients in the particular subset;
determining that the location of the particular patient is outside a predetermined distance threshold from the centroid and, responsive thereto, making the determination that the particular patient does not belong in the particular subset.

4. The method of claim 3, wherein the grouping further comprises:
assigning to each patient in the another subset a location along the set of Cartesian coordinates that corresponds with the recovery profile of the patient;
determining another centroid of the locations of the patients in the another subset;
determining the location of the particular patient is within another predetermined distance threshold from the another centroid and, responsive thereto, assigning the particular patient to the another subset.

5. The method of claim 1, further comprising:
making a determination, for a given patient of the plurality of patients who is in in a given subset of the plurality of subsets, and based at least in part upon the recovery profile of the particular patient, that the given patient does not belong in the particular subset;
determining that the given patient does not belong within any subset of the plurality of subsets; and
providing individualized medical care to the given patient.

6. The method of claim 1, further comprising predicting a need for at least one of staffing, resources, and equipment based at least in part upon the grouping together of the plurality of patients.

7. An apparatus structured to prescribing a treatment protocol for each of a plurality of patients, comprising:
a processor;
a storage, the storage having stored therein a number of routines which, when executed on the processor, cause the apparatus to perform a number of operations comprising:
at each of a plurality of times:
for each patient of the plurality of patients:
detecting a number of risk factors of the patient;
determining a triage risk index of the patient based at least in part upon an at least partial summation of at least a subset of the number of risk factors of the patient; and
determining a recovery rate of the patient based at least in part upon a rate of change in the triage risk index of the patient;
grouping together each patient from among the plurality of patients into a subset from among a plurality of subsets, each subset of the plurality of subsets corresponding with a phenotype from among a plurality of phenotypes, each phenotype from among the plurality of phenotypes corresponding with a treatment protocol from among a plurality of treatment protocols, the grouping being based at least in part upon a recovery profile of each patient in the subset; and
prescribing for each patient the treatment protocol that corresponds with the phenotype that corresponds with the subset to which the patient is assigned.

8. The apparatus of claim 7, wherein the grouping further comprises:
making a determination, for a particular patient of the plurality of patients who is in in a particular subset of the plurality of subsets, and based at least in part upon the recovery profile of the particular patient, that the particular patient does not belong in the particular subset;
assigning the particular patient to another subset of the plurality of subsets, the another subset being different from the particular subset, based at least in part upon the recovery profile of the particular patient; and
prescribing for the particular patient the treatment protocol that corresponds with the phenotype that corresponds with the another subset.

9. The apparatus of claim 8, wherein the grouping further comprises:
assigning to each patient in the particular subset a location along a set of Cartesian coordinates that corresponds with the recovery profile of the patient;
determining a centroid of the locations of the patients in the particular subset;
determining that the location of the particular patient is outside a predetermined distance threshold from the centroid and, responsive thereto, making the determination that the particular patient does not belong in the particular subset.

10. The apparatus of claim 9, wherein the grouping further comprises:
assigning to each patient in the another subset a location along the set of Cartesian coordinates that corresponds with the recovery profile of the patient;
determining another centroid of the locations of the patients in the another subset;
determining the location of the particular patient is within another predetermined distance threshold from the another centroid and, responsive thereto, assigning the particular patient to the another subset.

11. The apparatus of claim 7, wherein the operations further comprise:
making a determination, for a given patient of the plurality of patients who is in in a given subset of the plurality of subsets, and based at least in part upon the recovery profile of the particular patient, that the given patient does not belong in the particular subset;
determining that the given patient does not belong within any subset of the plurality of subsets; and
providing individualized medical care to the given patient.

12. The apparatus of claim 7, wherein the operations further comprise predicting a need for at least one of staffing, resources, and equipment based at least in part upon the grouping together of the plurality of patients.
